(19) United States
(12) Patent Application Publication
(10) Pub. No.: US 2004/0234482 A1
(43) Pub. Date: Nov. 25, 2004

(54) Cosmetic compositions comprising at least one crosslinked copolymer, at least one cyclodextrin and at least one surfactant, and uses thereof

(76) Inventors: Rainer Muller, Leopoldshafen (DE); Mireille Maubru, Chatou (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/796,182

(22) Filed: Mar. 10, 2004

Related U.S. Application Data
(60) Provisional application No. 60/454,623, filed on Mar. 17, 2003.

(30) Foreign Application Priority Data
Mar. 11, 2003 (FR) 03 02995

(51) Int. Cl. A61K 7/06; A61K 7/11

(52) U.S. Cl. 424/70.13

(57) ABSTRACT

Disclosed herein are compositions comprising, in an aqueous medium, such as in a physiologically acceptably aqueous medium, at least one surfactant, at least one ingredient chosen from cyclodextrins and derivatives thereof, and at least one crosslinked copolymer comprising at least one methacrylic acid and at least one C₁-C₄ alkyl acrylate unit, wherein the composition may further comprise at least one conditioner and the compositions are used as, for example, rinse-out products, such as for washing and/or conditioning keratinous materials. Also disclosed are methods of making these compositions, methods of treating keratinous material using the compositions, and the use of the at least one crosslinked copolymer comprising at least one methacrylic acid unit and at least one C₁-C₄ alkyl acrylate unit as an agent for suspending the complex formed by at least one ingredient chosen from cyclodextrins and derivatives thereof and at least one surfactant.

This application claims benefit of U.S. Provisional Application No. 60/454,623, filed Mar. 17, 2003.

Disclosed herein are compositions comprising, in a physiologically acceptable aqueous medium, at least one ingredient chosen from cyclodextrins and derivatives thereof, at least one surfactant, and at least one crosslinked copolymer comprising at least one methacrylic acid unit and at least one C₁-C₄ alkyl acrylate unit. The compositions disclosed herein can further comprise at least one conditioner. Further disclosed herein is the use of the at least one crosslinked copolymer comprising at least one methacrylic acid unit and at least one C₁-C₄ alkyl acrylate unit as an agent for suspending the complex formed from the at least one ingredient chosen from cyclodextrins and derivatives thereof and the at least one surfactant.

It is well known that hair that has been sensitized (i.e. damaged and/or embrittled) to varying degrees due to the action of atmospheric agents or mechanical or chemical treatments, such as dyeing, bleaching and/or permanent-waving, can often be difficult to disentangle and to style, and can lack softness.

It has already been recommended to use conditioners, such as insoluble conditioners, in compositions for washing or caring for keratinous materials, such as hair, in order to facilitate the disentangling of the hair and to give it softness, sheen and suppleness.

Given the insoluble nature of certain conditioners, for instance, silicones or oils, it is desirable to maintain the conditioners in a uniform dispersion in the medium without, however, causing a reduction in the viscosity and the detergent and foaming properties of the compositions. Conditioners, such as silicones and oils, should also be conveyed onto the treated keratinous materials in order to give them, depending on the application, softness, sheen and disentangling properties.

It is also known that products, for example, cosmetic products, having an iridescent, moiré or metallized appearance or effect are generally appreciated by consumers for their attractive appearance, giving the product a look of richness. The agents that can give the product this effect are nacreous agents or nacres generally comprising crystals that can remain dispersed in the compositions and can reflect light.

Long-chain ester derivatives are widely used to make compositions, such as cosmetic compositions, nacreous. However, these derivatives may have crystallization problems that can result in a change in the viscosity of the compositions over time.

Long-chain ether or thioether derivatives, such as those described in Patent Application Nos. EP 457 688 and WO 98/03155 are also known. However, these agents can opacify the compositions without, or without sufficiently, giving them a nacreous effect.

It has been found that, on account of their low density, these nacreous agents can often have the drawback of rising to the surface of the shampoo and forming a layer that consumers find to be unattractive.

Furthermore, in certain cases, these fatty-chain compounds can have the drawback of giving a laden feel to the hair and a lack of lightness and volume to the hairstyle.

Thus, there is still a need for novel nacreous agents that do not have at least one of the drawbacks mentioned above and that can also allow the use of insoluble conditioners, such as silicones.

Stabilizers, such as crosslinked acrylic polymers of the Carbopol type are frequently used to thicken and stabilize cosmetic compositions comprising insoluble conditioners. However, these stabilizers can have the drawback of reducing the cosmetic performance qualities of shampoos, and, for example, of making the hair more laden and more lank.

Cosmetic compositions, such as detergent compositions, comprising a crosslinked copolymer comprising at least one methacrylic acid unit and at least one alkyl acrylate unit as a stabilizer or suspension agent for water-insoluble ingredients, for instance, silicones or fatty substances, are known in the prior art. Such compositions have been described, for example, in Patent Application No. WO 01/76552. The foam qualities and the cosmetic properties that can be obtained with these compositions are still not sufficiently satisfactory.

In one embodiment, the present inventors have discovered that it is possible to formulate compositions, such as cosmetic compositions, for treating keratinous materials, for example, shampoos with a nacreous appearance, while at the same time having the desired aesthetic and cosmetic properties, by using, in these compositions, at least one ingredient chosen from cyclodextrins and derivatives thereof, at least one surfactant and at least one particular polymer.

Disclosed herein are compositions, such as cosmetic compositions, comprising, in an aqueous medium, for example, in a physiologically acceptable medium and further, for example, in a cosmetically acceptable medium, at least one surfactant, at least one ingredient chosen from cyclodextrins and derivatives thereof, and at least one crosslinked copolymer comprising at least one methacrylic acid unit and at least one C₁-C₄ alkyl acrylate unit.

In one embodiment, the presence of the at least one crosslinked copolymer comprising at least one methacrylic acid unit and at least one C₁-C₄ alkyl acrylate unit is not detrimental to the cosmetic properties of the composition, but can even improve at least one of these properties, such as with regard to the hair's smoothness, volume, and manageability.

In a further embodiment, the composition, as disclosed herein, may show generally very good homogeneity and good stability of the nacre, and also a viscosity that is satisfactory for application to keratinous materials. Additionally, there may be no phase separation.

For example, no uncontrolled graining-out or thickening of the composition may take place over time. Further, the compositions disclosed herein may have a non-runny, fondant texture. For example, the lather may be easy to rinse out.

Further disclosed herein is the use of at least one crosslinked copolymer comprising at least one methacrylic acid unit and at least one C₁-C₄ aklyl acrylate unit as an agent for suspending the insoluble complex formed by at least one cyclodextrin and at least one surfactant in a cosmetic composition, such as a washing and/or conditioning composition, comprising, in a physiologically acceptable aqueous medium, the at least one cyclodextrin and the at least one surfactant.

Also disclosed herein is a washing and/or conditioning process using such compositions.

Even further disclosed herein is the use of at least one crosslinked copolymer comprising at least one methacrylic acid unit and at least one C₁-C₄ aklyl acrylate unit in, or for the manufacture of, a cosmetic composition comprising at least one ingredient chosen from cyclodextrins and derivatives thereof and at least one surfactant.

Other subjects of the disclosure will become apparent on reading the description and the examples that follow.

As used herein, the term “unit” means the polymerized form of the monomer. For example a methacrylic acid unit is the polymerized form of methacrylic acid.

As used herein, the term “keratinous materials” means hair, eyelashes, eyebrows, skin, nails, mucous membranes and scalp. For example, a keratinous material is hair.

As used herein, the terms “nacreous agent” and “nacre” mean an agent that produces a nacreous, iridescent, moiré or metallized appearance or effect.

One of the characteristics of the present disclosure, for example, is the presence of at least one crosslinked copolymer comprising at least one methacrylic acid unit and at least one C₁-C₄ alkyl acrylate unit. As used herein, the crosslinked copolymer comprising at least one methacrylic acid unit and at least one C₁-C₄ alkyl acrylate unit means a crossklinked copolymer comprising at least one methacrylic acid unit and at least one alkyl acrylate unit, wherein the alkyl acrylate unit is chosen from C₁-C₄ alkyl acrylates.

In the crosslinked copolymers disclosed herein, the methacrylic acid unit can be present, for example, in an amount ranging from 20% to 80% by weight, such as from 25% to 70% by weight, and further such as from 35% to 60% by weight, relative to the total weight of the copolymer.

In the crosslinked copolymer disclosed herein, the alkyl acrylate unit can be present, for example, in an amount ranging from 15% to 80% by weight, such as from 25% to 75% by weight and further such as from 40% to 65% by weight, relative to the total weight of the copolymer. The alkyl acrylate unit can be chosen, for example, from methyl acrylate, ethyl acrylate and butyl acrylate. In one embodiment, the alkyl acrylate unit is ethyl acrylate.

The copolymer disclosed herein is partially or totally crosslinked with at least one standard crosslinking agent. The at least one crosslinking agent can be chosen, for example, from polyunsaturated compounds, such as polyethylenically unsaturated compounds. For example, these compounds can be chosen from polyalkenyl ethers of sucrose, polyalkenyl ethers of polyols, diallyl phthalates, divinylbenzene, allyl (meth)acrylate, ethylene glycol di(meth)acrylate, methylenebisacrylamide trimethylolpropane tri(meth)acrylate, diallyl itaconate, diallyl fumarate, diallyl maleate, zinc (meth)acrylate, castor oil derivatives and polyol derivatives manufactured from unsaturated carboxylic acids.

The at least one crosslinking agent that may also be used include, for example, unsaturated monomers comprising at least one reactive group capable of reacting with an unsaturation to form a crosslinked copolymer.

The content of the at least one crosslinking agent generally ranges from 0.01% to 5% by weight, for example, from 0.03% to 3% by weight, and further, for example, from 0.05% to 1% by weight, relative to the total weight of the copolymer.

According to one embodiment, the at least one crosslinked copolymer disclosed herein may be in the form of a dispersion in water. The number-average size of the copolymer particles in the dispersion generally ranges from 10 to 500 nm, for example, from 20 to 200 nm and further, for example, from 50 to 150 nm.

These copolymers are described, for example, in Patent Application No. WO 01/76552.

Use can be made, for example, of the crosslinked copolymers comprising at least one methacrylic acid unit and at least one ethyl acrylate unit in the form of an aqueous 30% dispersion manufactured and sold under the name Carbopol Aqua SF-1 by the company Noveon.

The concentration of the at least one crosslinked copolymer generally ranges from 0.01% to 10% by weight, and, for example, from 0.1% to 5% by weight, relative to the total weight of the composition.

The at least one ingredient chosen from cyclodextrin and derivatives thereof is chosen, for example, from oligosaccharides of formula:

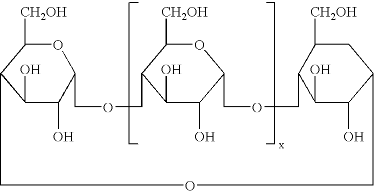

wherein x may be chosen from a number equal to 4 (which corresponds to α-cyclodextrin), to 5 (β-cyclodextrin) and to 6 (γ-cyclodextrin).

It is possible, for example, to use a β-cyclodextrin sold by the company Wacker under the name Cavamax W7 and a γ-cyclodextrin sold by the company Wacker under the name Cavamax W8.

The cyclodextrin derivatives are chosen, for example, from methylcyclodextrins, such as the methyl-β-cyclodextrin sold by the company Wacker under the name Cavasol W7.

As disclosed herein, the at least one ingredient chosen from cyclodextrins and derivatives thereof may be present in an amount ranging from 0.2% to 30% by weight, for example, from 1% to 15% by weight, and further, for example, from 1.5% to 10% by weight, relative to the total weight of the composition.

The compositions disclosed herein further comprise at least one surfactant, which is generally present in an amount ranging from 0.2% to 40% by weight, for example, from 1% to 35% by weight, and further, for example, from 1.5% to 30% by weight, relative to the total weight of the composition.

The at least one ingredient chosen from cyclodextrins and derivatives thereof and the at least one surfactant are present, for example, at a concentration that is effective to make the composition nacreous and/or to form an insoluble complex in the composition between the at least one ingredient chosen from cyclodextrins and derivatives thereof and the at least one surfactant.

The ratio of the at least one surfactant and the at least one ingredient chosen from cyclodextrins and derivatives thereof may range from 0.01:1 to 300:1, for example, from 0.1:1 to 100:1, and further, for example, from 0.3:1 to 25:1.

The at least one surfactant that is suitable for use herein may be of any nature, and can, for example, be soluble in water at room temperature. The at least one surfactant disclosed herein can be chosen from the following surfactants:

(i) Anionic Surfactants:

Thus, non-limiting examples of the anionic surfactants which can be used, alone or as mixtures, as disclosed herein, include: salts (such as alkaline salts, for example, sodium salts, ammonium salts, amine salts, amino alcohol salts and magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates and N-acyltaurates, alkyl and acyl groups of all of these various compounds comprising, for example, from 8 to 24 carbon atoms, and aryl groups chosen, for example, from phenyl and benzyl groups. Among the anionic surfactants which can also be used, non-limiting mention may also be made of fatty acid salts, such as salts of oleic, ricinoleic, palmitic and stearic acids, coconut oil acid and hydrogenated coconut oil acid; acyl lactylates wherein the acyl group comprises from 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and the salts thereof, such as those comprising from 2 to 50 ethylene oxide groups, and mixtures thereof.

Among the anionic surfactants, as disclosed herein, use can be made of alkyl sulphate salts, alkyl ether sulphate salts, and mixtures thereof.

(ii) Nonionic Surfactants:

The nonionic surfactants are compounds that are well known per se (see, for example, “Handbook of Surfactants” by M. R. Porter, published by Blackie & Son (Glasgow and London), 1991, pp. 116-178). Thus, the nonionic surfactants can be chosen, for example, from (non-limiting list) polyethoxylated fatty acids, polypropoxylated fatty acids and polyglycerolated fatty acids, alkylphenols, α-diols and alcohols, wherein all of these compounds comprise at least one fatty chain comprising, for example, from 8 to 18 carbon atoms, wherein it is possible for the number of ethylene oxide or propylene oxide groups to range, for example, from 2 to 50 and for the number of glycerol groups to range, for example, from 2 to 30. Non-limiting mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising from 1 to 5 glycerol groups, and, for example, from 1.5 to 4 glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides, such as (C₁₀-C₁₄)alkylamine oxides and N-acylaminopropylmorpholine oxides. In one embodiment, the nonionic surfactants disclosed herein are chosen from alkylpolyglycosides.

(iii) Amphoteric or Zwitterionic Surfactants:

Amphoteric or zwitterionic surfactants may, for example, be chosen from (non-limiting list), aliphatic secondary and tertiary amine derivatives wherein the at least one aliphatic group is chosen from linear and branched chains comprising from 8 to 18 carbon atoms and comprising at least one water-soluble anionic group (for example, carboxylate, sulphonate, sulphate, phosphate and phosphonate); mention may also be made of (C₈-C₂₀)alkylbetaines, sulphobetaines, (C₈-C₂₀)alkylamido(C₁-C₆)alkylbetaines and (C₈-C₂₀)alkylamido(C₁-C₆)alkylsulphobetaines.

Among the amine derivatives disclosed herein, non-limiting mention may be made of the products sold under the name Miranol, as described in U.S. Pat. Nos. 2,528,378 and 2,781,354 and classified in the CTFA dictionary, 3^{rd} edition, 1982 under the names amphocarboxy glycinates and amphocarboxy propionates and having the respective structures:

R₂—CON HCH₂CH₂—N⁺(R₃)(R₄)(CH₂COO⁻) (2)

wherein: R₂ is chosen from alkyl groups derived from an acid R₂—COOH present in hydrolysed coconut oil, heptyl, nonyl and undecyl groups, R₃ is a β-hydroxyethyl group and R₄ is a carboxymethyl group;

and

R₂—CONHCH₂CH₂—N(B)(C) (3)

wherein:

B is chosen from —CH₂CH₂OX′ groups, C is chosen from —(CH₂)ₙ—Y′ groups, wherein z=1 or 2,

X′ is chosen from —CH₂CH₂—COOH and a hydrogen atom,

Y′ is chosen from —COOH and —CH₂—CHOH—SO₃H,

R_{2′} is chosen from alkyl groups derived from an acid R_{2′}—COOH present in coconut oil or in hydrolysed linseed oil, alkyl groups, for example, C₇, C₉, C₁₁ and C₁₃ alkyl groups, a C₁₇ alkyl group and its iso form, and an unsaturated C₁₇ group.

For example, non-limiting mention may be made of cocoamphodiacetate sold under the trade name Miranol C2M concentrated NP by the company Rhodia Chimie.

(iv) Cationic Surfactants:

The cationic surfactants may, for example, be chosen from:

A) quaternary ammonium salts of general formula (XII) below:

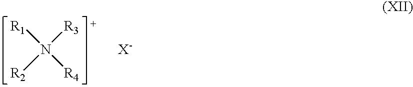

wherein X⁻ is an anion chosen from halides (such as chloride, bromide and iodide) and (C₂-C₆)alkyl sulphates, for example, methyl sulphate, phosphates, alkyl and alkylaryl sulphonates, anions derived from an organic acid, such as acetate and lactate, and

i) R₁, R₂, and R₃, which may be identical or different, are each chosen from linear and branched aliphatic groups comprising from 1 to 4 carbon atoms, and aromatic groups, such as aryl and alkylaryl. The aliphatic groups can comprise at least one hetero atom, such as oxygen, nitrogen, sulphur and halogens. The aliphatic groups are chosen, for example, from alkyl, alkoxy and alkylamide groups,

R₄ is chosen from linear and branched alkyl groups comprising from 16 to 30 carbon atoms.

The cationic surfactant can be chosen, for example, from cetyltrimethylammonium salts (for example, chloride).

ii) R₁ and R₂, which may be identical or different, can each be chosen from linear and branched aliphatic groups comprising from 1 to 4 carbon atoms, and aromatic groups, such as aryl and alkylaryl. The aliphatic groups can comprise at least one hetero atom, such as oxygen, nitrogen, sulphur and halogens. The aliphatic groups are chosen, for example, from alkyl, alkoxy, alkylamide and hydroxyalkyl groups comprising, for example, from 1 to 4 carbon atoms;

R₃ and R₄, which may be identical or different, can each be chosen from linear and branched alkyl groups comprising from 12 to 30 carbon atoms, wherein the alkyl groups comprise at least one functional group chosen from esters and amides.

R₃ and R₄ are chosen, for example, from (C₁₂-C₂₂)alkylamido(C₂-C₆)alkyl and (C₁₂-C₂₂)alkylacetate groups;

In one embodiment, the cationic surfactant is chosen, for example, from stearamidopropyldimethyl(myristyl acetate)ammonium salts (for example, chloride).

B) -quaternary ammonium salts of imidazolinium chosen, for example, from formula (XIII) below:

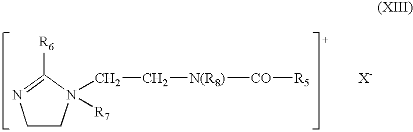

wherein R₅ is chosen from alkenyl and alkyl groups comprising from 8 to 30 carbon atoms, for example, fatty acid derivatives of tallow, R₆ is chosen from hydrogen, C₁-C₄-alkyl groups, and alkenyl and alkyl groups comprising from 8 to 30 carbon atoms, R₇ is chosen from C₁-C₄ alkyl groups, R₈ is chosen from a hydrogen atom and C₁-C₄ alkyl groups, and X⁻ is an anion chosen from halides, phosphates, acetates, lactates, alkyl sulphates, alkyl sulphonates and alkylaryl sulphonates. In one embodiment, R₅ and R₆ are chosen from a mixture of alkenyl and alkyl groups comprising from 12 to 21 carbon atoms, such as fatty acid derivatives of tallow, R₇ is methyl and R₈ is hydrogen. Such a product may be chosen, for example, from Quatemium-27 (CTFA 1997) and Quaternium-83 (CTFA 1997), which are sold under the names “Rewoquat” W75, W90, W75PG and W75HPG by the company Witco,

C) -diquaternary ammonium salts of formula (XIV):

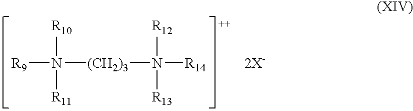

wherein R₉ is chosen from aliphatic groups comprising, for example, from 16 to 30 carbon atoms, R₁₀, R₁₁, R₁₂, R₁₃ and R₁₄, which may be identical or different, are each chosen from hydrogen and alkyl groups comprising from 1 to 4 carbon atoms, and X⁻ is an anion chosen from halides, acetates, phosphates, nitrates and methyl sulphates. Such diquaternary ammonium salts, for example, comprise propanetallowdiammonium dichloride.

D) -quaternary ammonium salts comprising at least one ester functional group, of formula (XV) below:

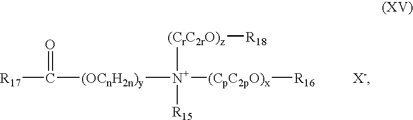

wherein:

R₁₅ is chosen from C₁-C₆ alkyl groups and C₁-C₆ hydroxyalkyl and dihydroxyalkyl groups;

R₁₆ is chosen from:

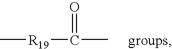

linear and branched, saturated and unsaturated C₁-C₂₂ hydrocarbon-based groups R₂₀, and

hydrogen,

R₁₈ is chosen from:

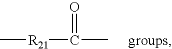

linear and branched, saturated and unsaturated C₁-C₆ hydrocarbon-based groups R₂₂, and

hydrogen,

R₁₇, R₁₉ and R₂₁, which may be identical or different, are each chosen from linear and branched, saturated and unsaturated C₇-C₂, hydrocarbon-based groups;

n, p and r, which may be identical or different, are each an integer ranging from 2 to 6;

y is an integer ranging from 1 to 10;

x and z, which may be identical or different, are each an integer ranging from 0 to 10;

X⁻ is an anion chosen from simple, complex, organic and inorganic anions; with the proviso that the sum x+y+z ranges from 1 to 15, that if x is 0, then R₁₆ is R₂₀ and that when z is 0, then R₁₈ is R₂₂.

For example, the ammonium salts of formula (XV), may be used, wherein:

R₁₅ is chosen from methyl and ethyl groups,

x and y are equal to 1;

z is equal to 0 or 1;

n, p and r are equal to 2;

R₁₆ is chosen from:

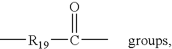

methyl, ethyl and C₁₄-C₂₂ hydrocarbon-based groups, and

hydrogen;

R₁₇, R₁₉ and R₂₁, which may be identical or different, are each chosen from linear and branched, saturated and unsaturated C₇-C₂₁ hydrocarbon-based groups;

R₁₈ is chosen from:

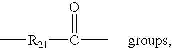

and

hydrogen;

Such compounds are sold, for example, under the names Dehyquart by the company Cognis, Stepanquat by the company Stepan, Noxamium by the company Ceca, and Rewoquat WE 18 by the company Degussa-Witco.

Among the quaternary ammonium salts that may be used, include, for example, cetyltrimethylammonium chloride and palmitamidopropyltrimethylammonium chloride sold under the name Varisoft PA TC by the company Degussa.

In one embodiment, the compositions comprise, for example, at least one anionic surfactant.

The anionic surfactants that may be used include, for example, sodium, triethanolamine and ammonium (C₁₂-C₁₄)alkyl sulphates, sodium, triethanolamine and ammonium (C₁₂-C₁₄)alkyl ether sulphates oxyethylenated with 2.2 mol of ethylene oxide, sodium cocoyl isethionate and sodium α-(C₁₄-C₁₆)olefin sulphonate, and mixtures thereof, with:

either an amphoteric surfactant, such as the amine derivatives known as disodium cocoamphodiacetate or sodium cocoamphopropionate sold, for example, by the company Rhodia Chimie under the trade name “Miranol® C2M CONCNP” as an aqueous solution comprising 38% active material, or under the name Miranol® C32;

or an amphoteric surfactant, such as alkylbetaines, for example, the cocobetaine sold under the name “Dehyton® AB 30” as an aqueous solution comprising 32% active material by the company Cognis, or such as (C₈-C₂₀)alkylamido(C₁-C₆)alkylbetaines, for example, Tegobetaine® F 50 sold by the company Degussa.

According to one embodiment, the cosmetic compositions may also comprise at least one agent for conditioning keratinous materials.

In one embodiment, when these compositions are applied to the hair, they can have good hair conditioning properties, for example, the treated hair can be smooth, can disentangle easily and may feel soft. The hair can have a natural, unladen appearance.

When the composition comprises at least one conditioner, the at least one conditioner may be chosen, for example, from synthetic oils, such as polyolefins, fluoro oils, fluoro waxes, fluoro gums, carboxylic acid esters, cationic polymers, silicones, mineral, plant and animal oils, ceramides and pseudoceramides, and mixtures thereof.

The polyolefins can, for example, be chosen from poly-α-olefins and, for instance:

of hydrogenated or nonhydrogenated polybutene type, and for example, hydrogenated or nonhydrogenated polyisobutene.

Isobutylene oligomers with a molecular weight of less than 1,000 and mixtures thereof with polyisobutylenes with a molecular weight of greater than 1,000 and, for example, ranging from 1,000 to 15,000, may be used.

Examples of poly-α-olefins that may be used, as disclosed herein, non-limiting mention may be made of the polyisobutenes sold under the name Permethyl 99 A, 101 A, 102 A, 104 A (n=16) and 106 A (n=38) by the company Presperse Inc., or the products sold under the name Arlamol HD (n=3) by the company ICI (n denoting the degree of polymerization),

of hydrogenated or nonhydrogenated polydecene type.

Such products are sold, for example, under the names Ethylflo by the company Ethyl Corp. and Arlamol PAO by the company ICI.

The mineral oils that may be used in the compositions disclosed herein can, for example, be chosen from:

hydrocarbons, such as hexadecane, and liquid paraffin;

The cationic polymers that may be used herein may be chosen, for example, from all those already known per se as improving the cosmetic properties of hair treated with detergent compositions, for example, those described in Patent Application No. EP-A-0 337 354 and in French Patent Applications Nos. FR-A-2 270 846, 2,383,660, 2,598 611, 2,470 596 and 2,519,863.

As used herein, the term “cationic polymer” means any polymer comprising cationic groups and/or groups that may be ionized into cationic groups.

Among the cationic polymers that may be used herein, non-limiting examples include, quaternary cellulose ether derivatives, such as the products sold under the name “JR 400” by the company Amerchol, cyclopolymers, for example, the dimethyldiallylammonium salt homopolymers and the copolymers of diallyldimethylammonium salts and of acrylamides, for instance the chlorides, sold under the names “Merquat 100”, “Merquat 550” and “Merquat S” by the company Nalco, cationic polysaccharides, and, for example, guar gums modified with 2,3-epoxypropyltrimethylammonium chloride, sold, for example, under the name “Jaguar C13S” by the company Meyhall, optionally crosslinked homopolymers and copolymers of a (meth)acryloyloxyethyltrimethylammonium salt, sold by the company Ciba as a 50% solution in mineral oil, under the trade names Salcare SC92 (crosslinked copolymer of methacryloyloxyethyltrimethylammonium chloride and of acrylamide) and Salcare SC95 (crosslinked homopolymer of methacryloyloxyethyltrimethylammonium chloride). It is also possible to use polymers comprising repeating units corresponding to the formula:

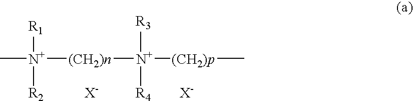

wherein R₁, R₂, R₃ and R₄, which may be identical or different, can each be chosen from alkyl and hydroxyalkyl groups comprising from 1 to 4 carbon atoms, n and p, which may be identical or different, are each an integer ranging from 2 to 20, and X⁻ is an anion derived from an acid chosen from mineral and organic acids.

The silicones that may be used herein, include, for example, polyorganosiloxanes that are insoluble in the composition and that may be in the form of oils, waxes, resins or gums.

The water-insoluble silicones are insoluble in water at a concentration of greater than or equal to 0.1% by weight in water at 25° C., i.e., they do not form a transparent isotropic solution.

The viscosity of the silicones is measured, for example, at 25° C. according to ASTM standard 445 Appendix C.

The organopolysiloxanes are defined in greater detail in Walter Noll's “Chemistry and Technology of Silicones” (1968) Academic Press. They can be volatile or nonvolatile.

When they are volatile, the silicones are chosen, for example, from those having a boiling point ranging from 60° C. and 260° C., and the followings:

(i) cyclic silicones comprising from 3 to 7 silicon atoms and, for example, from 4 to 5 silicon atoms. Such cyclic silicones include, for example, octamethylcyclotetrasiloxane sold under the name “Volatile Silicone 7207” by Union Carbide or “Silbione 70045 V 2” by Rhodia Chimie, decamethylcyclopentasiloxane sold under the name “Volatile Silicone 7158” by Union Carbide, and “Silbione 70045 V 5” by Rhodia Chimie, and mixtures thereof.

Mention may also be made of cyclocopolymers of the dimethylsiloxane/methylalkylsiloxane type, such as “Silicone Volatile FZ 3109” sold by the company Union Carbide, having the chemical structure:

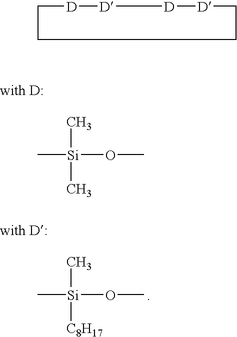

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones comprising from 2 to 9 silicon atoms and having a viscosity of less than or equal to 5×10⁻⁶ m²/s at 25° C. An example is decamethyltetrasiloxane sold, for example, under the name “SH 200” by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Nonvolatile silicones, for example, polyalkylsiloxanes, polyarylsiloxanes, polyalkylarylsiloxanes, silicone gums and resins, polyorganosiloxanes modified with organofunctional groups, and mixtures thereof, may, for example, be used.

The silicones can be chosen, for example, from polyalkylsiloxanes, among which mention may be made of polydimethylsiloxanes comprising trimethylsilyl end groups having a viscosity ranging from 5×10⁻⁶ m²/s to 2.5 m²/s at 25° C. and, for example, from 1×10⁻⁵ m²/s to 1 m²/s at 25° C.

Among the polyalkylsiloxanes, mention may be made, in a nonlimiting manner, of the following commercial products:

the oils of the Mirasil series sold by the company Rhodia Chimie, such as the oil Mirasil DM 500 000;

the oils of the 200 series from the company Dow Corning, such as DC200 with a viscosity of 60 000 cSt; and

the Viscasil oils from General Electric and certain oils of the SF series (SF 96, SF 18) from General Electric.

Mention may also be made of polydimethylsiloxanes comprising dimethylsilanol end groups (Dimethiconol according to the CTFA name), such as the oils of the 48 series from the company Rhodia Chimie.

In this category of polyalkylsiloxanes, mention may also be made of the products sold under the names “Abil Wax 9800 and 9801” by the company Degussa, which are poly(C₁-C₂₀)alkylsiloxanes.

The polyalkylarylsiloxanes can be chosen, for example, from linear and branched polydimethylmethylphenylsiloxanes and polydimethyldiphenylsiloxanes, with a viscosity ranging from 1×10⁻⁵ m²/s to 5×10⁻² m²/s at 25° C.

Among the polyalkylarylsiloxanes, mention may be made, by way of example, of the products sold under the following names:

the Mirasil DPDM oils from Rhodia Chimie;

the oils of the Rhodorsil 70 633 and 763 series from Rhodia Chimie;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000; and

certain oils of the SF series from General Electric, such as SF 1023, SF 1154, SF 1250, SF 1265.

The silicone gums that can be used herein include, for example, polydiorganosiloxanes having high number-average molecular masses ranging from 200,000 to 1,000,000, used alone or as a mixture in a solvent. The solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, and mixtures thereof.

Mention may be made, for example, of the following products:

polydimethylsiloxane,

polydimethylsiloxane/methylvinylsiloxane gums,

polydimethylsiloxane/diphenylsiloxane,

polydimethylsiloxane/phenylmethylsiloxane, and

polydimethylsiloxane/diphenylsiloxane/methylvinylsiloxane.

Products that can be used herein include the following mixtures, such as:

mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500,000, dissolved in the oil SF 1202 Silicone Fluid corresponding to decamethylcyclopentasiloxane; and

mixtures of two PDMSs of different viscosities and, for example, of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product, for example, comprises 15% SE 30 gum and 85% SF 96 oil.

The organopolysiloxane resins that can be used herein are chosen from crosslinked siloxane systems comprising the following units:

R₂SiO_{2/2}, R₃SiO_{1/2}, RSiO_{3/2} and SiO_{4/2} wherein R is chosen from hydrocarbon-based groups comprising from 1 to 16 carbon atoms and a phenyl group. For example, the products that may be used include those wherein R is chosen from C₁-C₄ lower alkyl groups, such as methyl, and a phenyl group.

Among these resins, mention may be made of the product sold under the name “Dow Corning 593” or those sold under the names “Silicone Fluid SS 4230 and SS 4267” by the company General Electric, which are silicones of dimethyl/trimethyl siloxane structure.

Mention may also be made of the trimethyl siloxysilicate type resins sold, for example, under the names X22-4914, X21-5034 and X21-5037 by the company Shin-Etsu.

The organomodified silicones that can be used herein include silicones as defined above and comprising in their structure at least one organofunctional group attached via a hydrocarbon-based group.

Among the organomodified silicones, mention may be made of polyorganosiloxanes comprising:

at least one group chosen from polyethyleneoxy and polypropyleneoxy groups optionally comprising at least one alkyl group chosen from C₆-C₂₄ alkyl groups, such as the products known as dimethicone copolyol sold by the company Dow Corning under the name DC 1248 or the oils Silwet L 722, L 7500, L 77 and L 711 by the company Union Carbide, and the (C₁₂)alkylmethicone copolyol sold by the company Dow Corning under the name Q2 5200;

optionally substituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups include, for example, at least one aminoalkyl group chosen from C₁-C₄ aminoalkyl groups;

thiol groups, such as the products sold under the names “GP 72 A” and “GP 71” from Genesee;

alkoxylated groups, such as the product sold under the name “Silicone Copolymer F-755” by SWS Silicones and Abil Wax 2428, 2434 and 2440 by the company Degussa;

hydroxylated groups, such as the polyorganosiloxanes comprising at least one hydroxyalkyl functional group, described in French Patent Application No. FR-A-85 16334;

acyloxyalkyl groups, such as the polyorganosiloxanes described in U.S. Pat. No. 4,957,732;

anionic groups of carboxylic type, such as in the products described in Patent No. EP 186 507 from the company Chisso Corporation, or of alkylcarboxylic type, such as those present in the product X-22-3701 E from the company Shin-Etsu; 2-hydroxyalkyl sulphonate; 2-hydroxyalkyl thiosulphate, such as the products sold by the company Degussa under the names “Abil S201” and “Abil S255”;

hydroxyacylamino groups, such as the polyorganosiloxanes described in Patent Application No. EP 342 834. Non-limiting mention may be made, for example, of the product Q2-8413 from the company Dow Corning.

As disclosed herein, it is also possible to use silicones comprising a polysiloxane portion and a portion comprising a non-silicone organic chain, wherein one of the two portions constitutes the main chain of the polymer, and the other is grafted onto the main chain. These polymers are described, for example, in Patent Applications Nos. EP-A-412 704, EP-A-412 707, EP-A-640 105, WO 95/00578, EP-A-582 152 and WO 93/23009 and U.S. Pat. Nos. 4,693,935, 4,728,571 and 4,972,037. These polymers can be, for example, anionic or nonionic.

Such polymers are, for example, copolymers that can be obtained by free-radical polymerization starting with a monomer mixture comprising:

a) 50% to 90% by weight of tert-butyl acrylate;

b) 0% to 40% by weight of acrylic acid;

c) 5% to 40% by weight of silicone macromer of formula:

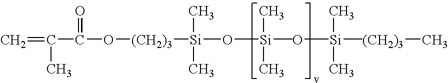

wherein v is a number ranging from 5 to 700; and the weight percentages are calculated relative to the total weight of the monomers.

Other examples of grafted silicone polymers are, for example, polydimethylsiloxanes (PDMS) onto which are grafted, via a connecting chain unit of thiopropylene type, mixed polymer units of poly(meth)acrylic acid type and of polyalkyl (meth)acrylate type and polydimethylsiloxanes (PDMS) onto which are grafted, via a connecting chain unit of thiopropylene type, polymer units of polyisobutyl (meth)acrylate type.

As disclosed herein, the silicones can also be used in the form of emulsions.

The polyorganosiloxanes that can be used herein include, for example:

nonvolatile silicones chosen from the family of polyalkylsiloxanes comprising trimethylsilyl end groups, such as oils having a viscosity ranging from 0.2 m²/s to 2.5 m²/s at 25° C., such as the oils of the DC200 series from Dow Corning, for example, the oils with a viscosity of 60,000 cSt, of the Mirasil DM series and, for example, the oil Mirasil DM 500 000 sold by the company Rhodia Chimie or the silicone oil AK 300.000 from the company Wacker, polyalkylsiloxanes comprising dimethylsilanol end groups, such as dimethiconols and polyalkylarylsiloxanes, such as the oil Mirasil DPDM sold by the company Rhodia Chimie; and

polysiloxanes comprising amine groups, such as amodimethicones and trimethylsilylamodimethicones.

As disclosed herein, the compounds of ceramide type may be chosen, for example, from natural and synthetic ceramides and glycoceramides and pseudoceramides and neoceramides.

Compounds of ceramide type are described, for example, in Patent Applications Nos. DE 4 424 530, DE 4 424 533, DE 4 402 929, DE 4 420 736, WO 95/23807, WO 94/07844, EP-A-0 646 572, WO 95/16665, FR-2 673 179, EP-A-0 227 994, WO 94/07844, WO 94/24097 and WO 94/10131, the teachings of which are incorporated herein by reference.

Compounds of ceramide type that may be used include, for example:

2-N-linoleoylaminooctadecane-1,3-diol,

2-N-oleoylaminooctadecane-1,3-diol,

2-N-palmitoylaminooctadecane-11,3-diol,

2-N-stearoylaminooctadecane-1,3-diol,

2-N-behenoylaminooctadecane-1,3-diol,

2-N-[2-hydroxypalmitoyl]aminooctadecane-1,3-diol,

2-N-stearoylaminooctadecane-1,3,4-triol, such as N-stearoylphytosphingosine,

2-N-palmitoylaminohexadecane-1,3-diol

bis(N-hydroxyethyl-N-cetyl)malonamide,

N-(2-hydroxyethyl)-N-(3-cetyloxy-2-hydroxypropyl)cetylamide; and

N-docosanoyl-N-methyl-D-glucamine

and mixtures of these compounds.

As disclosed herein, the at least one conditioner may be present in an amount ranging from 0.001% to 10% by weight, for example, from 0.005% to 5% by weight, and, further, for example, from 0.01% to 3% by weight, relative to the total weight of the composition.

The physiologically acceptable medium can, for example., comprise water and a mixture of water and at least one cosmetically or dermatologically acceptable solvent, such as monoalcohols, polyalcohols and glycol ethers, which may be used alone or as a mixture. The water can be present in an amount ranging from 30% to 98% by weight, and, for example, from 50% to 98% by weight, relative to the total weight of the composition.

Non-limiting mention may be made of monoalcohols, such as ethanol and isopropanol, polyalcohols, such as diethylene glycol and glycerol, glycol ethers, and alkyl ethers of glycol or of diethylene glycol.

In one embodiment, the composition disclosed herein can further comprise at least one additive chosen from sequestering agents, softeners, foam modifiers, dyes, other nacreous agents, hydrating agents, antidandruff and antiseborrheic agents, other suspension agents, fatty acids comprising at least one chain chosen from linear and branched C₁₆-C₄₀ chains, hydroxy acids, electrolytes, thickeners, fatty acid esters, fragrances, preserving agents, sunscreens, proteins, vitamins and provitamins, polymers and any other additive conventionally used in cosmetics.

The at least one additive disclosed herein, may be present in an amount ranging, for example, from 0 to 40% by weight, relative to the total weight of the composition. The precise amount of each additive depends on its nature and is readily determined by a person of ordinary skill in the art.

Needless to say, a person of ordinary skill in the art will take care to select the optional compound(s) to be added to the composition disclosed herein, such that the advantageous properties intrinsically associated with the composition are not, or are not substantially, adversely affected by the envisaged addition.

The compositions disclosed herein may be in a form chosen from gels, milks, creams, more or less thickened lotions and mousses.

The compositions disclosed herein may be used for treating keratinous materials, such as hair, skin, eyelashes, eyebrows, nails, lips and scalp. In one embodiment, the keratinous material is hair.

The compositions may also be used for washing and cleansing keratinous materials, such as hair and skin.

The compositions disclosed herein are, for example, used as products for washing, caring for, conditioning and maintaining the hairstyle and for shaping keratinous materials, such as hair.

The compositions disclosed herein may be in a form chosen from shampoos, rinse-out and leave-in conditioners, compositions for permanent-waving, relaxing, dyeing and bleaching the hair, compositions to be applied before and after dyeing, bleaching, permanent-waving and relaxing the hair and between the two steps of a permanent-waving and relaxing operation. For example, the compositions are washing and foaming compositions for hair and skin.

For instance, the compositions disclosed herein are foaming detergent compositions, such as shampoos, shower gels and bubble baths. In this embodiment, the compositions comprise at least one detergent surfactant.

The at least one detergent surfactant may be chosen from the anionic, amphoteric, nonionic, zwitterionic and cationic surfactants described above.

The minimum amount of surfactant is that which is just sufficient to give the final composition a satisfactory foaming power and/or detergent power.

Thus, as disclosed herein, the at least one detergent surfactant may be present in an amount ranging from 3% to 30% by weight, for example, from 6% to 25% by weight, and further, for example, from 8% to 20% by weight, relative to the total weight of the composition.

The foaming power of the compositions disclosed herein is characterized by a foam height, which is generally greater than 75 mm and, for example, greater than 100 mm, measured according to the modified Ross-Miles method (NF T 73-404/ISO 696).

The modifications to the method are as follows:

The measurement is performed at a temperature of 22° C. with osmosed water. The concentration of the solution is 2 g/l. The drop height is 1 m. The amount of composition which is dropped is 200 ml. The 200 ml of composition fall into a measuring cylinder 50 mm in diameter and comprising 50 ml of the test composition. The measurement is carried out 5 minutes after stopping the flow of the composition.

The compositions disclosed herein may also be in a form chosen from rinse-out and leave-in conditioners, permanent-waving, relaxing, dyeing and bleaching compositions, rinse-out compositions, to be applied before or after dyeing, bleaching, permanent-waving and relaxing the hair and between the two steps of permanent-waving and hair-relaxing operations.

When the composition is in the form of a conditioner optionally to be rinsed out, it can, for example, comprise at least one cationic surfactant present in an amount ranging, for example, from 0.1% to 10% by weight, and, further, for example, from 0.5% to 5% by weight, relative to the total weight of the composition.

The compositions disclosed herein may also be in the form of washing compositions for the skin, and, for example, in the form of bath and shower solutions, gels, and makeup-removing products.

The compositions disclosed herein mayalso be in the form of aqueous and aqueous-alcoholic lotions for skincare and/or haircare.

The cosmetic compositions disclosed herein may be in the form of gels, milks, creams, emulsions, lotions and mousses and may be used for skin, nails, eyelashes, lips and, for example, hair. The compositions can be, for example, in a liquid form.

The compositions may be packaged in various forms, such as in vaporizers, pump-dispenser bottles and aerosol containers in order to be able to apply the composition in a vaporized form or in the form of a mousse. Such packaging forms are indicated, for example, when it is desired to obtain a spray, a lacquer or a mousse for treating the hair.

Further disclosed herein, is a cosmetic process for treating keratinous materials such as the hair, comprising applying to the keratinous material the composition as defined above, optionally followed by rinsing with water after an optional leave in time.

The disclosure will now be illustrated more fully with the aid of non-limiting examples that follow. In the text hereinbelow, AM means Active Material.

### EXAMPLES 1 AND 2

Two shampoos comprising the following compositions were prepared:

| Composition | Example 1 | Example 2 |
|---|---|---|
| Sodium lauryl ether sulphate comprising | 11.8 g AM | 15.5 g AM |
| 2.2 mol of ethylene oxide | | |
| Cocoylbetaine | 1.5 g AM | — |
| Cocoylamidopropylbetaine | — | 2.4 g AM |
| Methacrylic acid/ethyl acrylate copolymer as | 10 g | 11 g |
| an aqueous emulsion comprising 30% active | | |
| material, sold by the company Noveon | | |
| Cyclodextrin, sold under the name | 1.5 g | 1.5 g |
| Cavamax W7 by the company Wacker | | |
| Polydimethylsiloxane of viscosity | 1.5 g | — |
| 500 000 cSt, sold under the name Mirasil | | |
| DM 500 000 by the company Rhodia | | |
| Polydimethylsiloxane of viscosity | — | 2.7 g |
| 300 000 cSt, sold under the name DC200 | | |
| Fluid 300 000 by the company Dow Corning | | |
| Hydroxyethylcellulose crosslinked with | 0.4 g | — |
| epichlorohydrin, quaternized with | | |
| trimethylamine, sold under the name JR 400 | | |
| by the company Amerchol | | |
| Guar gum modified with 2, 3- | — | 0.1 g |
| epoxypropyltrimethylammonium chloride, | | |
| sold under the name Jaguar C13 S by the | | |
| company Rhodia | | |
| Preserving agents, fragrance | qs | qs |
| pH agent qs | pH 7 | pH 7 |
| Demineralized water qs | 100 g | 100 g |

### EXAMPLES 3 AND 4

The following shampoo compositions were prepared:

| | Example 3 | Example 4 |
|---|---|---|
| Polyquaternium-10 | 0.8 g | 0.8 g |
| Carbopol Aqua SF 1 | 2 g | 3 g |
| Cyclodextrin (Cavamax from Wacker) | 2 g | 2 g |
| Sodium laureth sulphate (70% AM) | 16 g | 16 g |
| Cocamidopropylbetaine (30% AM) | 8 g | 8 g |
| PEG-60 hydrogenated castor oil | 0.2 g | 0.2 g |
| Fragrance | 0.7 g | 0.7 g |
| Olea europaea | 0.01 g | 0.01 g |
| Citrus limonum | 0.01 g | 0.01 g |
| DMDM hydantoin/sodium methylparaben | 0.45 g | 0.45 g |
| pH agent (tartaric acid/citric acid/aqueous | pH 5.5 | pH 5.5 |
| ammonia) qs | | |
| Demineralized water qs | 100 g | 100 g |
| Appearance | nacreous | nacreous |
| Storage test | Stable for | Stable for 2 |
| | 2 months at | months at |
| | 45° C. | 45° C. |

### EXAMPLE 5

The following shampoo composition was prepared:

| | |
|---|---|
| Demineralized water | qs 100 g |
| Sodium laureth sulphate (70% AM) | 22.2 g |
| Carbopol Aqua SF 1 | 5 g |
| Sodium hydroxide | 0.19 g |
| Cocobetaine (30% AM) | 9 g |
| Sodium cetearyl sulphate (90% AM) | 0.75 g |
| Cyclodextrin, sold under the name Cavamax W7 by | 2.5 g |
| the company Wacker | |
| Propylene glycol | 0.1 g |
| Piroctone olamine | 0.5 g |
| Dimethicone (Mirasil DM 500 000 from the | 2 g |
| company Rhodia) | |
| Vitamins | 0.2 g |
| Extract of lemon and apple juice | 0.6 g |
| DMDM hydantoin/paraben/phenoxyethanol | 0.5 g |
| Fragrance | qs |
| pH agent qs | pH 6.3 |
| Appearance | nacreous |
| Storage test | Stable for 2 months |
| | at 45° C. |

What is claimed is:

1. A composition comprising, in an aqueous medium,
at least one surfactant,
at least one ingredient chosen from cyclodextrins and derivatives thereof, and
at least one crosslinked copolymer comprising at least one methacrylic acid unit and at least one C₁-C₄ alkyl acrylate unit.

2. The composition according to claim 1, wherein the at least one ingredient chosen from cyclodextrins and derivates thereof is chosen from α-cyclodextrin, β-cyclodextrin and γ-cyclodextrin, and derivatives thereof.

3. The composition according to claim 2, wherein the at least one ingredient chosen from cyclodextrins and derivatives thereof is chosen from β-cyclodextrin and γ-cyclodextrin.

4. The composition according to claim 1, wherein the at least one ingredient chosen from cyclodextrins and derivatives thereof is present in an amount ranging from 0.2% to 30% by weight, relative to the total weight of the composition.

5. The composition according to claim 4, wherein the at least one ingredient chosen from cyclodextrins and derivatives thereof is present in an amount ranging from 1% to 15% by weight, relative to the total weight of the composition.

6. The composition according to claim 1, wherein the at least one surfactant and the at least one ingredient chosen from cyclodextrins and derivatives thereof are present in concentrations that are effective to form an insoluble complex in the composition and/or to make the composition nacreous.

7. The composition according to claim 1, wherein in the at least one crosslinked copolymer, the at least one methacrylic acid unit is present in an amount ranging from 20% to 80% by weight, relative to the total weight of the copolymer.

8. The composition according to claim 7, wherein in the at least one crosslinked copolymer, the at least one methacrylic acid unit is present in an amount ranging from 25% to 70% by weight, relative to the total weight of the copolymer.

9. The composition according to claim 8, wherein in the at least one crosslinked copolymer, the at least one methacrylic acid unit is present in an amount ranging from 35% to 60% by weight, relative to the total weight of the copolymer.

10. The composition according to claim 1, wherein, in the at least one crosslinked copolymer, the at least one C₁-C₄ alkyl acrylate unit is present in an amount ranging from 15% to 80% by weight, relative to the total weight of the copolymer.

11. The composition according to claim 10, wherein, in the at least one crosslinked copolymer, the at least one C₁-C₄ alkyl acrylate unit is present in an amount ranging from 25% to 75% by weight, relative to the total weight of the copolymer.

12. The composition according to claim 11, wherein, in the at least one crosslinked copolymer, the at least one C₁-C₄ alkyl acrylate unit is present in an amount ranging from 40% to 65% by weight, relative to the total weight of the copolymer.

13. The composition according to claim 1, wherein, in the at least one crosslinked copolymer, the at least one C₁-C₄ alkyl acrylate unit is chosen from methyl acrylate, ethyl acrylate and butyl acrylate.

14. The composition according to claim 13, wherein the at least one C₁-C₄ alkyl acrylate unit is ethyl acrylate.

15. The composition according to claim 1, wherein the at least one crosslinked copolymer is crosslinked with at least one polyethylenically unsaturated crosslinking agent.

16. The composition according to claim 15, wherein the at least one polyethylenically unsaturated crosslinking agent is present in an amount ranging from 0.01% to 5% by weight, relative to the total weight of the at least one crosslinked copolymer.

17. The composition according to claim 16, wherein the at least one polyethylenically unsaturated crosslinking agent is present in an amount ranging from 0.03% to 3% by weight, relative to the total weight of the at least one crosslinked copolymer.

18. The composition according to claim 17, wherein the at least one polyethylenically unsaturated crosslinking agent is present in an amount ranging from 0.05% to 1%, by weight relative to the total weight of the at least one crosslinked copolymer.

19. The composition according to claim 1, wherein the at least one crosslinked copolymer is present in an amount ranging from 0.01% and 10% by weight, relative to the total weight of the composition.

20. The composition according to claim 19, wherein the at least one crosslinked copolymer is present in an amount ranging from 0.1% and 5% by weight, relative to the total weight of the composition.

21. The composition according to claim 1, wherein the at least one surfactant is chosen from anionic surfactants.

22. The composition according to claim 1, wherein the at least one surfactant is present in an amount ranging from 0.2% to 40% by weight, relative to the total weight of the composition.

23. The composition according to claim 22, wherein the at least one surfactant is present in an amount ranging from 1% to 35% by weight, relative to the total weight of the composition.

24. The composition according to claim 1, wherein the composition further comprises at least one conditioner.

25. The composition according to claim 24, wherein the at least one conditioner is chosen from polyolefins, fluoro oils, fluoro waxes, fluoro gums, carboxylic acid esters, silicones, cationic polymers, mineral, plant and animal oils, ceramides and pseudoceramides, and mixtures thereof.

26. The composition according to claim 25, wherein the polyolefins are chosen from poly-α-olefins.

27. The composition according to claim 24, wherein the at least one conditioner is present in an amount ranging from 0.001% to 10% by weight, relative to the total weight of the composition.

28. The composition according to claim 27, wherein the at least one conditioner is present in an amount ranging from 0.005% to 5% by weight, relative to the total weight of the composition.

29. The composition according to claim 28, wherein the at least one conditioner is present in an amount ranging from 0.01% to 3% by weight, relative to the total weight of the composition.

30. The composition according to claim 1, wherein the composition is in a form chosen from gels, milks, creams, lotions, and mousses.

31. The composition according to claim 1, wherein the composition is a foaming detergent composition.

32. The composition according to claim 31, wherein the foaming detergent composition is chosen from shampoos, shower gels and bubble bath compositions.

33. The composition according to claim 1, wherein the composition is chosen from rinse-out and leave-in conditioning compositions, permanent-waving, relaxing, dyeing and bleaching compositions, and compositions to be applied before or after dyeing, bleaching, permanent-waving or relaxing hair, and between the two steps of a permanent-waving or hair-relaxing operation.

34. A composition comprising, in an aqueous medium, at least one crosslinked copolymer comprising at least one methacrylic acid unit, at least one C₁-C₄ alkyl acrylate unit, and at least one polyethylenically unsaturated crosslinking agent.

35. A composition comprising, in an aqueous medium,
at least one surfactant,
at least one ingredient chosen from cyclodextrins and derivatives thereof, and
at least one crosslinked copolymer polymerized from at least one methacrylic acid, at least one C₁-C₄ alkyl acrylate, and at least one polyethylenically unsaturated crosslinking agent.

36. A cosmetic process for treating a keratinous material, comprising applying to the keratinous material, a composition comprising, in an aqueous medium,
at least one surfactant,
at least one ingredient chosen from cyclodextrins and derivatives thereof,
and at least one crosslinked copolymer comprising at least one methacrylic acid unit and at least one C₁-C₄ alkyl acrylate unit, and
optionally rinsing the keratinous material with water.

37. The process according to claim 36, wherein the keratinous material is hair.

38. A method for manufacturing a cosmetic composition comprising, including in the composition at least one crosslinked copolymer comprising at least one methacrylic acid unit and at least one C₁-C₄ alkyl acrylate unit, wherein the composition comprises at least one surfactant and at least one ingredient chosen from cyclodextrins and derivatives thereof.

39. A method for suspending a complex formed by at least one compound chosen from cyclodextrins and derivatives thereof and at least one surfactant, comprising, including in a cosmetic composition, at least one crosslinked copolymer comprising at least one methacrylic acid unit and at least one C₁-C₄ alkyl acrylate unit as an agent for the suspending.

40. The method according to claim 38, wherein the cosmetic composition is chosen from washing and conditioning compositions.
